(19) United States
(12) Patent Application Publication
(10) Pub. No.: US 2011/0218170 A1
(43) Pub. Date: Sep. 8, 2011

(54) USE OF 2'-DEOXY-4'-THIOCYTIDINE AND ITS ANALOGUES AS DNA HYPOMETHYLATING ANTICANCER AGENTS

(75) Inventors: Jaideep Thottassery, Birmingham, AL (US); Kamal N. Tiwari, Birmingham, AL (US); William B. Parker, Birmingham, AL (US); John A. Secrist, III, Birmingham, AL (US)

(73) Assignee: Southern Research Institute, Birmingham, AL (US)

(21) Appl. No.: 12/715,811

(22) Filed: Mar. 2, 2010

(51) Int. Cl. A61K 31/7068; A61P 35/00

(52) U.S. Cl. 514/49; 514/43

(57) ABSTRACT

Compounds represented by the formulae:

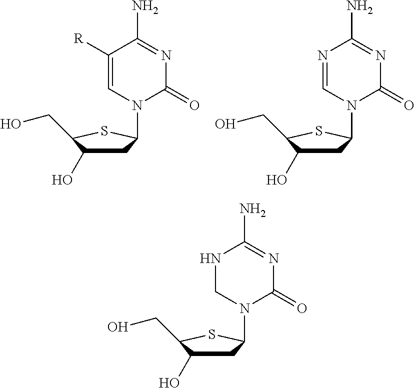

wherein R is individually selected from the group consisting of H, aliphatic acyl, aromatic acyl group, fluoro, chloro, bromo, iodo, alkoxy, alkyl, haloalkyl, alkenyl, haloalkenyl, alkynyl, amino, monoalkylamino, dialkylamino, cyano, aryl and nitro; pharmaceutically acceptable salts thereof, prodrugs thereof, solvates thereof and mixtures thereof; are used as inhibitors of DNA methyltransferase and for treating patients suffering from diseases resulting from or related to aberrant DNA methylation such as myelodysplastic syndromes and other cancers.

### FEDERALLY SPONSORED RESEARCH AND DEVELOPMENT

This invention was supported by Grant CA 34200 from the National Institutes of Health and the US Government has certain rights in the invention.

### TECHNICAL FIELD

The present disclosure relates to certain cytidine nucleosides that are useful as inhibitors of DNA methyltransferases (DNMTs). The present disclosure relates to methods of using these compounds to treat diseases in which inhibition of DNA methylation results in beneficial effects.

### BACKGROUND OF THE INVENTION

Cancer is considered to be a leading cause of death in the United States with one of every four Americans likely to be diagnosed with the disease. Even though significant advances have occurred in the treatment of cancer, it still remains a major health concern. A considerable amount of research over the years has led to the identification of many drug compounds that kill tumor cells and inhibit tumor progression. Some of this research has resulted in finding FDA-approved treatments for patients afflicted with various cancers although complete cures are rare. Furthermore compounds that are found to exhibit cytotoxicity are quite often not selective against tumor cells. Therefore, efforts continue at an ever increasing rate in view of the extreme difficulty in uncovering promising anticancer treatments and there still remains room for improved drugs that are effective for the desired treatment, while at the same time exhibiting reduced adverse side effects.

Inhibition of DNA methylation using cytidine/deoxycytidine analogs is now being recognized as another strategy to combat cancer cells and is proving to be effective as indicated by the recent approvals of 5-azacytidine (5-azaCyd) and 5-aza-2′-deoxycytidine (5-azadCyd) in myelodysplastic syndromes (MDS) and certain leukemias. Studies suggest that the inhibition of DNA cytosine-5 methylation leads to the re-expression of silenced tumor suppressors which contributes to the beneficial effects of these drugs. However inhibition of DNA synthesis and other toxicities of these compounds represent major drawbacks in the clinic.

### SUMMARY OF THE INVENTION

The uses of 2′-deoxycytidine analogues with 4′-thio and other modifications, which are DNA hypomethylators, by virtue of their ability to inhibit human DNA methyltransferase (DNMT1), are disclosed in this application. In particular, the present disclosure relates to a method for inhibiting DNA methylation in cells of patients by administering to the patient at least one compound represented by the formulae:

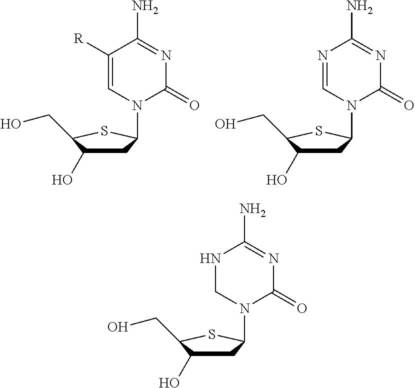

wherein R is selected individually from the group consisting of H, aliphatic acyl, aromatic acyl, halo, alkoxy, alkyl, haloalkyl, alkenyl, haloalkenyl, alkynyl, amino, monoalkylamino, dialkylamino, cyano, aryl and nitro; a pharmaceutically acceptable salt thereof, a prodrug thereof, solvate thereof and mixtures thereof; in an amount effective for inhibiting DNA methylation.

A still further aspect of the present disclosure relates to a method for treating a patient suffering from aberrant DNA methylation related diseases which comprises administering to said patient an effective amount of at least one of the above disclosed compounds. Another aspect of the present invention relates to a method for preventing or treating a mammalian host at risk of developing cancer, or one who has been diagnosed with cancer, which comprises administering to said host an effective amount of at least one compound represented by the formulae; a pharmaceutically acceptable salt thereof; a prodrug thereof or a solvate thereof.

Still other objects and advantages of the present disclosure will become readily apparent by those skilled in the art from the following detailed description of preferred embodiments, wherein it is shown simply by way of illustration of the best mode contemplated. As will be realized the disclosure is capable of other and different embodiments, and its several details are capable of modifications in various obvious respects, without departing from the disclosure. Accordingly, the description is to be regarded as illustrative in nature and not as restrictive.

### BRIEF DESCRIPTION OF THE DRAWINGS

The foregoing and other objects, features, and advantages of the invention are apparent from the following detailed description taken in conjunction with the accompanying drawings in which:

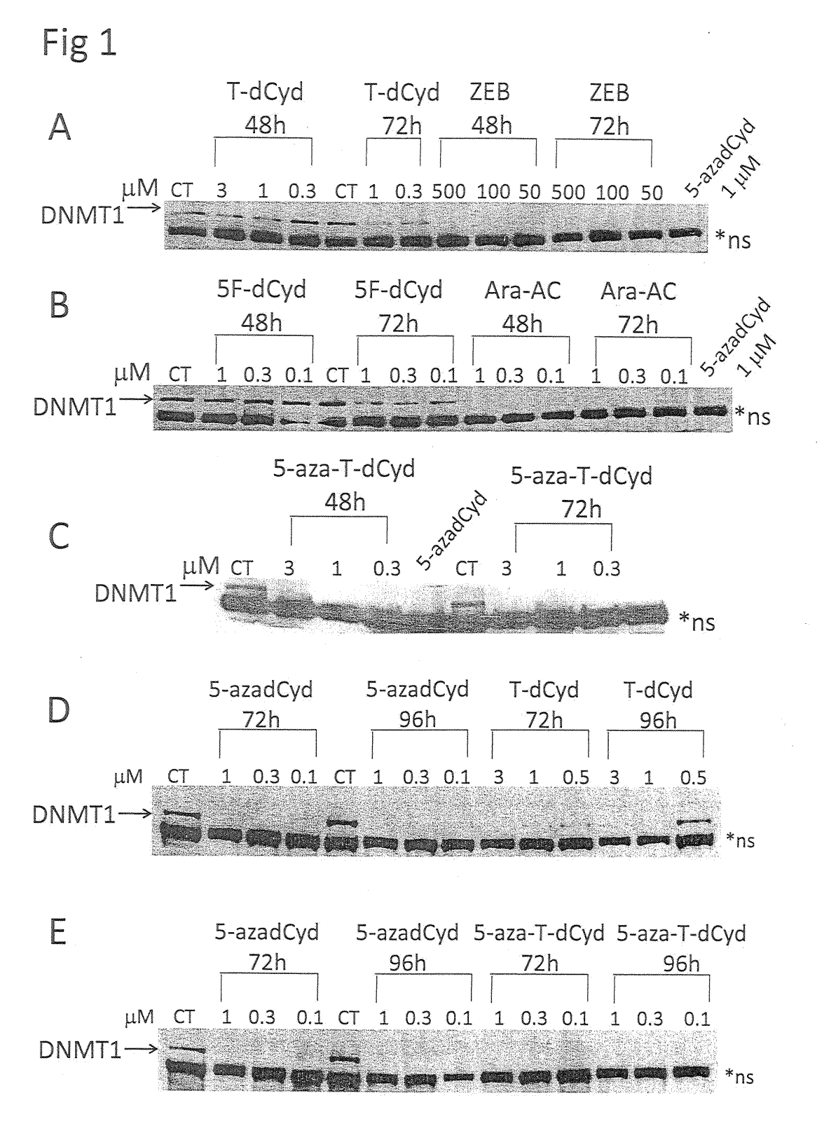

a) FIGS. 1 (A, B, C, D and E) illustrates the effect of 4′-thio-2′-deoxycytidine (T-dCyd) and 5-aza-4′-thio-2′-deoxycytidine (5-aza-T-dCyd) on DNMT1 protein levels in comparison with that of zebularine (ZEB), 5-aza-2′-deoxycytidine (5-azadCyd), 5-fluoro-2′-deoxycytidine (5F-dCyd) and ara-AC (Fazarabine, i.e. 5-aza-arabinofuranosylcytosine) in KG1a myeloid leukemia cells.

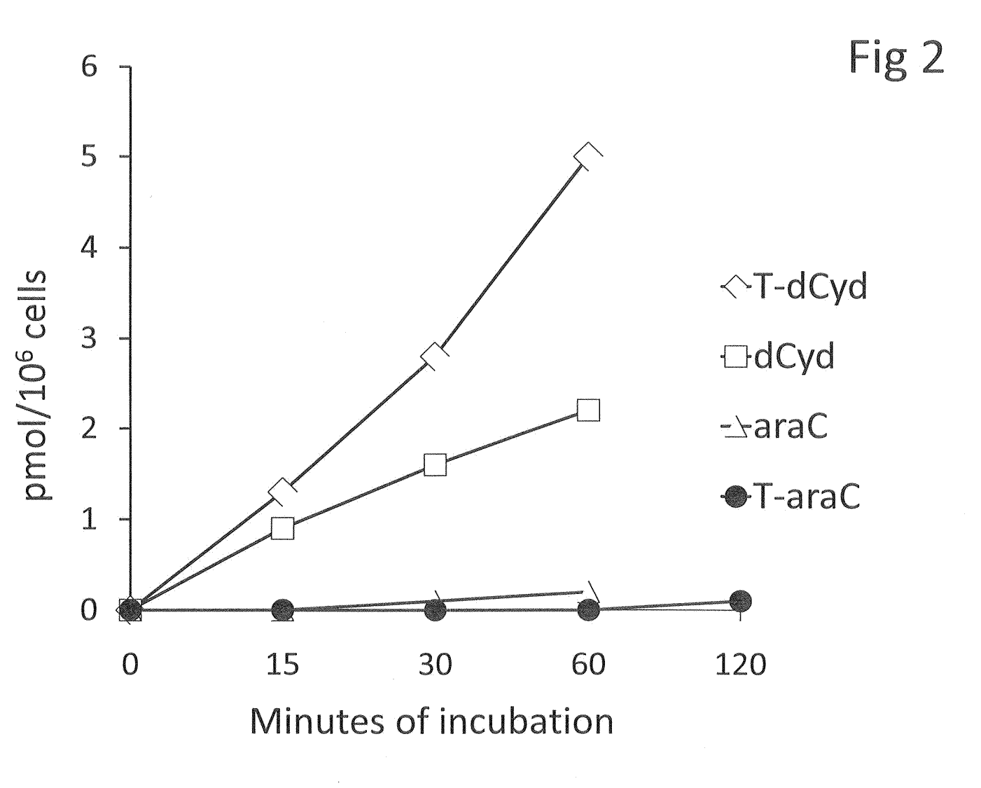

b) FIG. 2 compares the incorporation of T-dCyd into DNA with that of the natural 2′-deoxycytidine, and also with that of araC (arabinofuranosylcytosine) and T-araC (4′-thio-arabinofuranosylcytosine).

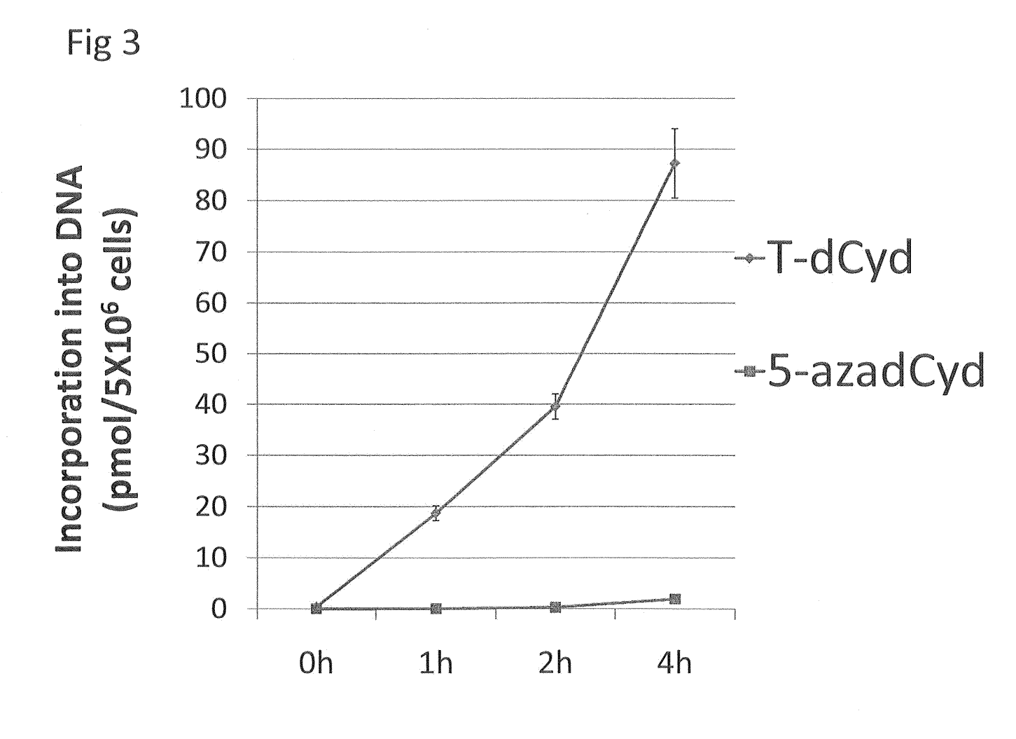

c) FIG. 3 compares the incorporation of T-dCyd into DNA versus that of 5-azadCyd.

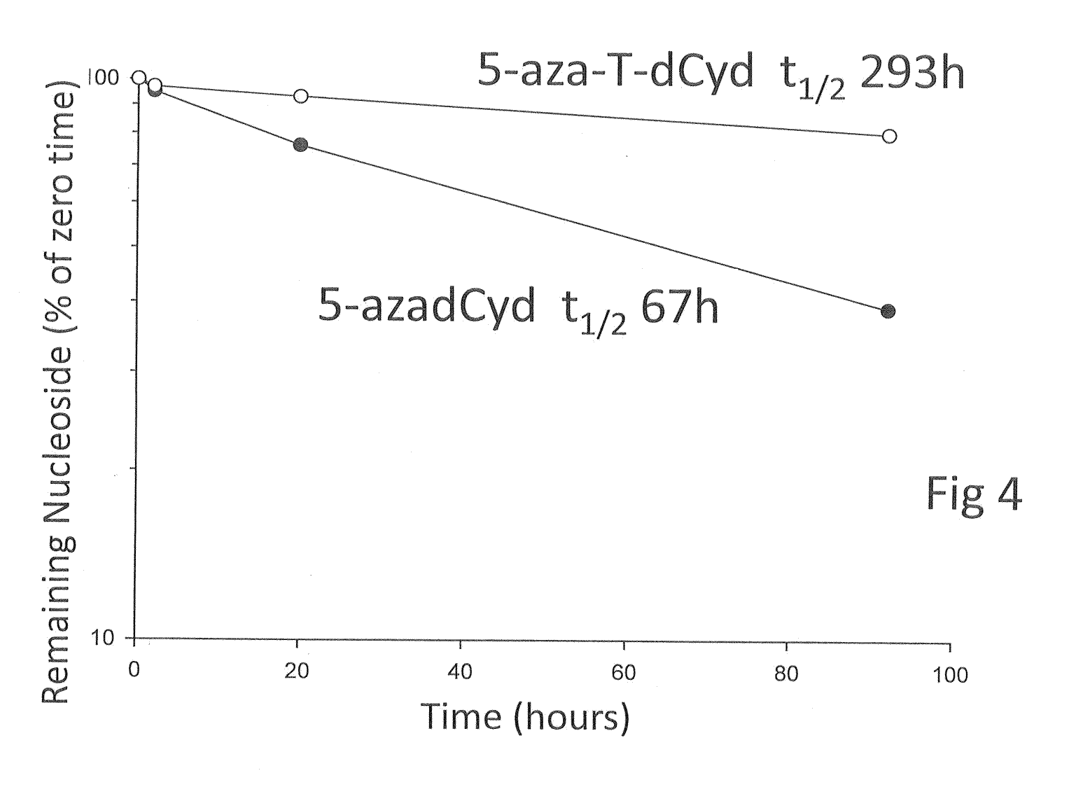

d) FIG. 4 illustrates the stability of 5-aza-T-dCyd in phosphate buffered saline in comparison with that of 5-azadCyd.

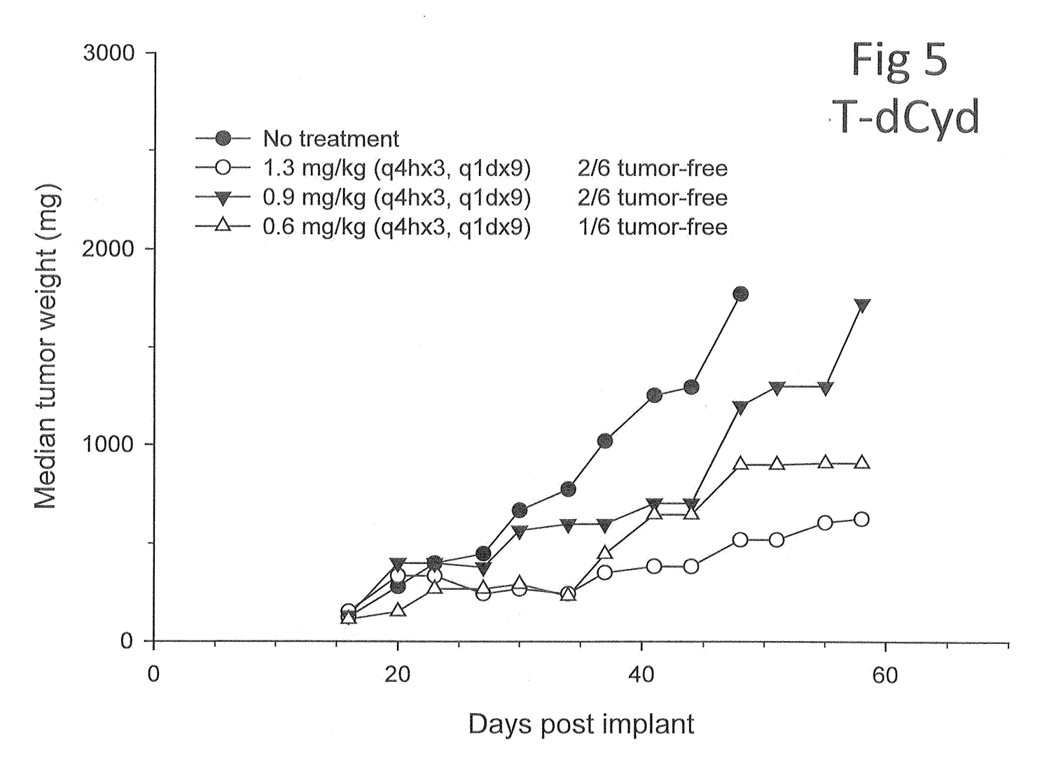

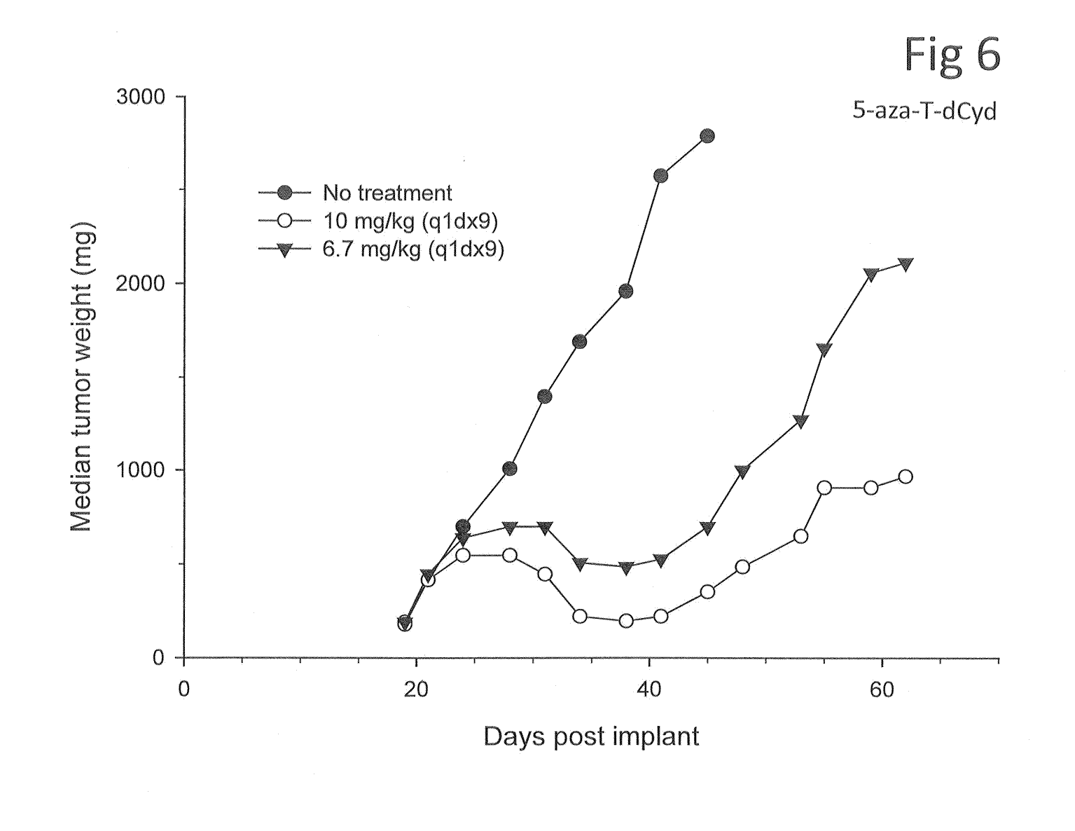

e) FIG. 5 and FIG. 6 show results of anti-tumor activity of T-dCyd and 5-aza-T-dCyd in in vivo tumor models, respectively.

### BEST AND VARIOUS MODES FOR CARRYING OUT THE INVENTION

In particular, the present disclosure relates to use of compounds represented by the following formulae:

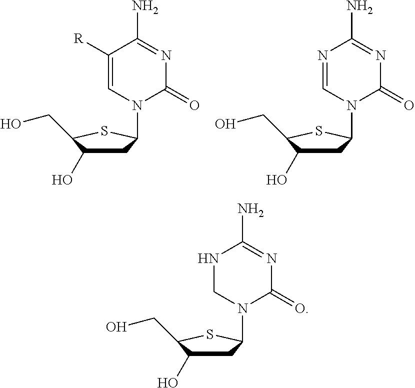

wherein R is individually selected from the group consisting of H, aliphatic acyl, aromatic acyl, halo, alkoxy, alkyl, haloalkyl, alkenyl, haloalkenyl, alkynyl, amino, monoalkylamino, dialkylamino, cyano, aryl and nitro; a pharmaceutically acceptable salt thereof, a prodrug thereof, solvates and mixtures thereof.

Listed below are definitions of various terms used to describe this invention. These definitions apply to the terms as they are used throughout this specification, unless otherwise limited in specific instances, either individually or as part of a larger group.

Typical aliphatic acyl groups contain 1 to 6 carbon atoms and include formyl, acetyl and propionyl.

Typical aromatic acyl groups include unsubstituted and alkyl substituted aromatic groups containing 7 to 10 carbon atoms in the aromatic ring. When substituted the alkyl group typically contains 1-6 carbon atoms. Typical aromatic acyl groups include benzoyl and para-toluoyl.

The term “alkyl” refers to straight or branched chain unsubstituted hydrocarbon groups of typically 1 to 22 carbon atoms, more typically 1 to 8 carbon atoms, and even more typically 1 to 4 carbon atoms.

Examples of suitable alkyl groups include methyl, ethyl and propyl. Examples of branched alkyl groups include isopropyl and t-butyl.

The alkoxy group typically contains 1 to 6 carbon atoms. Suitable alkoxy groups typically contain 1-6 carbon atoms and include methoxy, ethoxy, propoxy and butoxy.

Suitable haloalkyl groups typically contain 1-6 carbon atoms and can be straight or branched chain and include Cl, Br, F or I, substituted alkyl groups including the above specifically disclosed alkyl groups.

Suitable alkenyl groups typically contain 2-6 carbon atoms and include ethenyl and propenyl.

Suitable haloalkenyl groups typically contain 1-6 carbon atoms and include Cl, Br, F or I, substituted alkenyl groups including the above specifically disclosed alkenyl groups.

Suitable alkynyl groups typically contain 1-6 carbon atoms and include ethynyl and propynyl.

Suitable monoalkylamino groups contain 1-6 carbon atoms and include monomethylamino, monoethylamino, mono-isopropylamino, mono-n-propylamino, mono-isobutyl-amino, mono-n-butylamino and mono-n-hexylamino. The alkyl moiety can be straight or branched chain.

Suitable dialkylamino groups contain 1-6 carbon atoms in each alkyl group. The alkyl groups can be the same or different and can be straight or branched chain. Examples of some suitable groups are dimethylamino, diethylamino, ethylmethylamino, dipropylamino, dibutylamino, dipentylamino, dihexylamino, methylpentylamino, ethylpropylamino and ethylhexylamino.

Examples of halo groups are Cl, F, Br and I.

The term “aryl” refers to monocyclic or bicyclic aromatic hydrocarbon groups having 6 to 12 carbon atoms in the ring portion, such as phenyl, naphthyl, biphenyl, and diphenyl groups, each of which may be substituted such as with a halo or alkyl group.

It is of course understood that the compounds of the present disclosure relate to all optical isomers and stereo-isomers at the various possible atoms of the molecule, unless specified otherwise.

The compounds according to this disclosure may form prodrugs at hydroxyl or amino functionalities using alkoxy, amino acids, etc. groups as the prodrug forming moieties. For instance, the hydroxymethyl position may form mono-, di- or triphosphates and again these phosphates can form prodrugs.

Preparations of such prodrug derivatives are discussed in various literature sources (examples are: Alexander et al., J. Med. Chem. 1988, 31, 318; Aligas-Martin et al., PCT WO pp/41531, p. 30). The nitrogen function converted in preparing these derivatives is one (or more) of the nitrogen atoms of a compound of the disclosure.

“Pharmaceutically acceptable salts” refer to derivatives of the disclosed compounds wherein the parent compound is modified by making acid or base salts thereof. The compounds of this disclosure form acid and base addition salts with a wide variety of organic and inorganic acids and bases and includes the physiologically acceptable salts which are often used in pharmaceutical chemistry. Such salts are also part of this disclosure. Typical inorganic acids used to form such salts include hydrochloric, hydrobromic, hydroiodic, nitric, sulfuric, phosphoric, hypophosphoric and the like. Salts derived from organic acids, such as aliphatic mono and dicarboxylic acids, phenyl substituted alkonic acids, hydroxyalkanoic and hydroxyalkandioic acids, aromatic acids, aliphatic and aromatic sulfonic acids, may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, β-hydroxybutyrate, butyne-1,4-dioate, hexyne-1,4-dioate, cabrate, caprylate, chloride, cinnamate, citrate, formate, fumarate, glycollate, heptanoate, hippurate, lactate, malate, maleate, hydroxymaleate, malonate, mandelate, mesylate, nicotinate, isonicotinate, nitrate, oxalate, phthalate, teraphthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzene-sulfonate, p-bromobenzenesulfonate, chlorobenzenesulfonate, ethanesulfonate, 2-hydroxyethanesulfonate, methanesulfonate, naphthalene-1-sulfonate, naphthalene-2-sulfonate, p-toleunesulfonate, xylenesulfonate, tartarate, and the like.

Bases commonly used for formation of salts include ammonium hydroxide and alkali and alkaline earth metal hydroxides, carbonates, as well as aliphatic and primary, secondary and tertiary amines, aliphatic diamines. Bases especially useful in the preparation of addition salts include sodium hydroxide, potassium hydroxide, ammonium hydroxide, potassium carbonate, methylamine, diethylamine, and ethylene diamine.

“Solvates” refers to the compound formed by the interaction of a solvent and a solute and includes hydrates. Solvates are usually crystalline solid adducts containing solvent molecules within the crystal structure, in either stoichiometric or nonstoichiometric proportions.

The term “comprising” (and its grammatical variations) as used herein is used in the inclusive sense of “having” or “including” and not in the exclusive sense of “consisting only of.” The terms “a” and “the” as used herein are understood to encompass the plural as well as the singular.

### Compound Synthesis

Compounds of the present disclosure can be prepared according to methods described in Tiwari K N, Cappellacci L, Montgomery J A, Secrist J A, III. Synthesis and anti-cancer activity of some novel 5-azacytosine nucleosides. Nucleosides Nucleotides Nucleic Acids 2003, 22:2161-2170; and Secrist J A, III, Tiwari K N, Riordan J M, Montgomery J A. Synthesis and biological activity of 2′-deoxy-4′-thio pyrimidine nucleosides. J. Med. Chem. 1991, 34:2361-2366; and U.S. Pat. No. 5,591,722 to Montgomery et al. and assigned to Southern Research Institute, the assignee of this application and European Patent 0 421 777 B1 to Walker et al; entire disclosures of which are incorporated herein by reference.

By way of example, the following scheme using 5-aza-T-dCyd is presented to further facilitate an understanding of this disclosure.

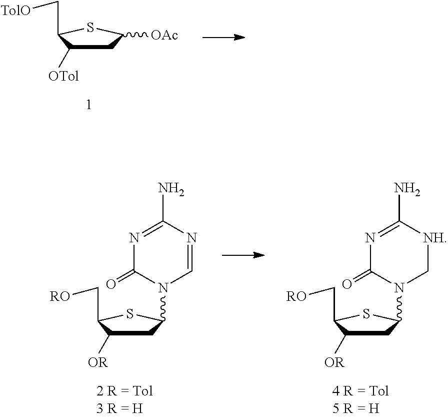

The present disclosure is concerned with inhibiting DNMTs and with treating patients afflicted with diseases related to or resulting from aberrant DNA methylation. In brief, it has been observed according to the present disclosure that T-dCyd and 5-aza-T-dCyd can deplete human DNMT1 in cancer cells (FIGS. 1A, 1C, 1D and 1E). It has also been observed according to the present disclosure that T-dCyd is inserted into replicating DNA (FIG. 2 and FIG. 3). Therefore, T-dCyd is readily activated to its triphosphate T-dCTP (4′-thio-2′-deoxycytidine triphosphate), which is a good substrate for DNA polymerase mediated incorporation, and DNA polymerases also readily extend the chain after incorporation. It has also been observed that the T-dCMP formed in T-dCyd treated cells will not be deaminated (Table 1), and therefore, intracellular levels of unwanted metabolites that could inhibit thymidylate synthase would be diminished. Structural studies have also shown that DNA bearing 4′-thio modifications are only subtly altered. Thus 4′-thionucleosides are bioisosteric with respect to the natural 4′-oxonucleosides and have other advantages such as a stable glycosyl bond and increased metabolic stability against cellular enzymes. Furthermore, it has been observed according to the present disclosure that T-dCyd and 5-aza-T-dCyd are efficacious in in vivo tumor models (FIG. 5 and FIG. 6). Studies suggest that the inhibition of DNA cytosine-5 methylation and the re-expression of silenced tumor suppressors contribute to the beneficial effects of these drugs. However inhibition of DNA synthesis and other toxicities of FDA approved DNA hypomethylators represent major drawbacks in the clinic. In contrast we have found that T-dCyd exhibited very little toxicity at nanomolar doses although it is robustly incorporated into DNA and markedly depletes DNMT1. Collectively therefore, the data according to the present disclosure suggest that these analogues could serve as excellent choices for inhibitors of DNA methylation with better properties for cancer therapy and chemoprevention than existing approved agents.

FIGS. 1A and 1B depicts results from an experiment wherein KG1a human myeloid leukemia cells were treated with T-dCyd or with ZEB, 5F-dCyd, 5-azadCyd or ara-AC. Cells were incubated with drugs for the indicated times and analyzed by western blot. (*ns indicates a non-specific band used as a loading control). The results clearly show that a 72 hour exposure at low non-toxic doses of T-dCyd was capable of reducing DNMT1, comparable to levels obtained with 5F-dCyd. Strikingly, 5-aza-T-dCyd completely depleted DNMT1 at similar exposures for as little as 48 h (FIG. 1C). In similar experiments T-araC did not deplete DNMT1 (not shown) as is the case with araC. Interestingly, ara-AC was also capable of depleting DNMT1 similar to 5-azadCyd (FIG. 1B), which indicates that the arabinose sugar is not fundamentally detrimental to this effect. However since ara-AC is a chain terminator and therefore its DNA synthesis inhibition effects are likely to limit the doses at which it can be administered, which can ultimately affect the efficiency of hypomethylation. We have also extended these findings and demonstrate that T-dCyd induced complete depletion of DNMT1 at 96 h at the 1 and 3 μM doses in these cells and also at 72 h at the 3 μM dose (FIG. 1D). Also shown in FIG. 1E is the complete depletion of DNMT1 by 5-aza-T-dCyd at doses as low as 0.1 μM when exposed for 72 or 96 h.

The metabolism of T-dCyd in human cells has also been evaluated, and it has been found that it is activated to the 5′-triphosphate of T-dCyd (T-dCTP), which is readily used as a substrate for DNA synthesis. In particular, FIG. 2 refers to an experiment where CEM leukemia cells were incubated with 100 nM of [5-³H]T-dCyd, [5-³H]dCyd, [5-³H]araC or [5-³H]T-araC and the incorporation of compound into the DNA was determined. As seen in this figure, more T-dCyd was incorporated into the DNA than dCyd, which indicated that T-dCyd was activated and incorporated into DNA without disruption of DNA synthesis. In contrast much less araC (or T-araC) was incorporated into the DNA. Although araCTP (or T araCTP) are also good substrates for DNA polymerases, they are known to inhibit subsequent DNA chain elongation and thereby inhibit DNA replication. This indicates that arabinose sugars would not be a good strategy in the design of DNA hypomethylators. The concentration of T-dCyd or araC required to inhibit CEM cell growth by 50% after 72 hours of incubation was 2.2 or 0.006 μM, respectively. These results indicate that, unlike araC, a considerable amount of T-dCyd can be incorporated into the DNA without cell cytotoxicity. Similar results were observed with 4′-thio-thymidine (T-dThd), which supported the conclusions that were obtained with T-dCyd (i.e. 4′-thio-dNTPs are readily used substrates by DNA polymerases involved in DNA replication without inhibition of subsequent DNA synthesis by these polymerases).

Whereas in FIG. 2 data demonstrating that T-dCyd was incorporated into the DNA as well or better than the natural nucleoside dCyd in CEM murine leukemia cells was presented, FIG. 3 compares T-dCyd incorporation into DNA versus that of the currently clinically used 5-azadCyd in KG1a human myeloid leukemia cells. The data indicate that only a moderate amount of 5-azadCyd is incorporated into DNA after 4 h of incubation at 300 nM in these cells. In contrast T-dCyd at the same dose is rapidly incorporated to very high levels in these cells similar to our findings in CEM cells. This provides further evidence that a considerable amount of T-dCyd can be incorporated into the DNA at low doses and indicates that 4′-thio-dCTP is readily used as a substrate by DNA polymerases involved in DNA replication without inhibition of subsequent DNA synthesis by DNA polymerases.

The metabolites that were produced in CEM cells treated with either [5-³H]dCyd or [5-³H]T-dCyd were also determined (Table 1). Cells were treated with 100 nM of each compound for 1 hour and the medium and cells were extracted to determine the major metabolites that were formed. The medium was analyzed by reverse phase HPLC to measure the parent compound, deaminated product, or [³H]-water. The acid-soluble extract was analyzed by strong anion exchange HPLC to measure the intracellular concentration of nucleotide metabolites. The radioactivity in the acid-insoluble fraction represents the amount of compound that was incorporated into DNA. As shown in Table 1 below, 55% of the radioactivity associated with dCyd was converted to water, which indicated that most of the dCMP (deoxycytidine monophosphate) that was formed from dCyd was deaminated to dUMP (deoxyuridine monophosphate) by dCMP deaminase and then used for TMP synthesis, instead of being converted to dCTP (deoxycytidine triphosphate). (Note: Because dCyd was labeled at the 5 position, the [³H] was removed from the molecule by thymidylate synthase in the process of adding a methyl group to this position. Accordingly, there will be no radioactivity in thymidine nucleotides generated from [5-³H]dCyd).

TABLE 1
Metabolism of dCyd or T-dCyd in CEM cells

| [5-³H]- | [³H] in | [³H] in | | | |
|---|---|---|---|---|---|
| labelled | parent | deaminated | [³H] in | [³H] in | [³H] in |
| compound | compound | compound | water | triphosphate | DNA |
| | (percent of total) | | | | |
| dCyd | 38 | 0 | 55 | 4 | 3 |
| T-dCyd | 90 | 0 | 0 | 2 | 8 |

Importantly, none of the radioactivity associated with T-dCyd was recovered as water in the medium, T-dUrd (4′-thiodeoxyuridine) in the medium (data not shown), or as T-dUMP (4′-thiodeoxyuridine monophosphate) in the acid-soluble pool (data not shown). This result indicated that T-dCMP was not a good substrate for dCMP deaminase. This result is significant, because it indicates that T-dCyd analogs will not be converted to dUMP analogs by this particular metabolic route of dCyd, and therefore, intracellular levels of unwanted metabolites that could inhibit thymidylate synthase would be diminished. In sum, therefore it is reasonable to conclude, that at the doses used, the metabolism of T-dCyd and its analogs are unlikely to cause thymidylate synthase inhibition and its resultant effects on DNA synthesis.

Importantly, some of the [³H]-water generated from [5-³H]dCyd (Table 1), albeit a small percentage, could also be from proton abstraction during methylation at position 5 by DNMT1 after DNA incorporation. However, it is of interest that none of the [5-³H] from T-dCyd appeared in water, which suggests that neither are its metabolites good substrates for thymidylate synthase, nor is T-dCyd a good substrate for DNMT catalysis. In sum, the results suggest that T-dCyd in DNA is not methylated or is poorly methylated, which is consistent with the results obtained with purified M. Hha1 methyltransferase and T-dCyd in DNA.

One of the primary problems of the currently clinically used DNA hypomethylators 5-aza-dCyd and 5-azacytidine is the rapid degradation of these compounds by ring opening of the 5-azacytosine ring in aqueous solutions. Whether a 4′-thio modification would enhance the stability of 5-azacytosine relative to the 4′-oxonucleoside was tested (FIG. 4). In side-by-side experiments, the compounds were dissolved in PBS (phosphate buffered solution, pH 7.4) at a concentration of 100 μM. A sample was removed from each solution 2, 20, and 90 hours after addition of the drug and the concentrations of 5-aza-T-dCyd or 5-aza-dCyd were determined by reverse phase HPLC. The half-lives of 5-aza-dCyd and 5-aza-T-dCyd were determined to be 67 and 293 hours, respectively (FIG. 4), which indicated that 5-aza-T-dCyd was 5-fold more stable than 5-aza-dCyd in PBS, a result that could have large practical significance in treating patients.

Preliminary in vivo evaluation of the anti-tumor activity of T-dCyd and 5-aza-T-dCyd in mice was also conducted (FIG. 5 and FIG. 6). T-dCyd was tested against human lung NCI-H23 (FIG. 5), and also in DLD-1 colon tumor xenografts (not shown). At a dose of 1.3 mg/kg (MTD_{0.65}) with a 9 day treatment there was substantial growth inhibition in NCI-H23 tumors with 2/6 complete cures (note: MTD was 2 mg/kg). However the 9-day treatment schedule is a classical schedule for a cytotoxic anticancer agent. T-dCyd as a potential DNA hypomethylating agent has not been evaluated in tumor models. The 0.9 mg/kg (MTD_{0.45}) and the 0.6 mg/kg (MTD_{0.3}) doses also suggested some encouraging in vivo activity in a 9 day treatment schedule (FIG. 5).

Much better results were obtained with 5-aza-T-dCyd, where the compound substantially inhibited the growth of the tumors (FIG. 6). Interestingly the tumor growth was not inhibited immediately, but tumor size decreased several days after administration of the drug was terminated (day 28). This pattern of tumor growth during treatment with 5-aza-T-dCyd suggests that the compound is not acting as a typical cytotoxic, but is instead having a delayed effect presumably due to the inhibition of DNA methylation. Again, the schedule in this experiment (q1dx9) was also not designed based on the supposed mechanism of action of this agent, but was designed based on a classical cytotoxic nucleoside analog, as in the case of T-dCyd. Based on the presumed mechanism of action (inhibition of DNA methylation) these results suggest that better schedules could be identified that could further inhibit tumor growth, without toxicity. This type of compound might best be used in combination with other chemotherapy as a maintenance therapy. Thus, in the clinic one could envision treatment first with cytotoxic agents to kill a large percentage of rapidly dividing tumor cells, followed by treatment with non-toxic doses of hypomethylating agents for longer time periods. In a similar vein one could envisage treatment with non-toxic DNA hypomethylating agents in chemoprevention settings.

### Formulations

The compounds of the present disclosure can be administered by any conventional means available for use in conjunction with pharmaceuticals, either as individual therapeutic agents or in a combination of therapeutic agents. They can be administered alone, but generally are administered with a pharmaceutical carrier selected on the basis of the chosen route of administration and standard pharmaceutical practice. The compounds can also be administered in conjunction with other therapeutic agents such as interferon (IFN), interferon α-2a, interferon α-2b, consensus interferon (CIFN), ribavirin, amantadine, remantadine, interleukine-12, ursodeoxycholic acid (UDCA), and glycyrrhizin or other agents contemplated for the desired use of the compounds such as cancer.

The pharmaceutically acceptable carriers described herein, for example, vehicles, adjuvants, excipients, or diluents, are well-known to those who are skilled in the art. Typically, the pharmaceutically acceptable carrier is chemically inert to the active compounds and has no detrimental side effects or toxicity under the conditions of use. The pharmaceutically acceptable carriers can include polymers and polymer matrices.

The compounds of this disclosure can be administered by any conventional method available for use in conjunction with pharmaceuticals, either as individual therapeutic agents or in a combination of therapeutic agents.

The dosage administered will, of course, vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of active ingredient can be expected to be about 0.001 to 1000 milligrams (mg) per kilogram (kg) of body weight, with the preferred dose being 0.1 to about 30 mg/kg.

Dosage forms (compositions suitable for administration) typically contain from about 1 mg to about 500 mg of active ingredient per unit. In these pharmaceutical compositions, the active ingredient will ordinarily be present in an amount of about 0.5-95% weight based on the total weight of the composition.

The active ingredient can be administered orally in solid dosage forms, such as capsules, tablets, and powders, or in liquid dosage forms, such as elixirs, syrups and suspensions. It can also be administered parenterally, in sterile liquid dosage forms. The active ingredient can also be administered intranasally (nose drops) or by inhalation of a drug powder mist. Other dosage forms are potentially possible such as administration transdermally, via patch mechanism or ointment.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

The compounds of the present disclosure, alone or in combination with other suitable components, can be made into aerosol formulations to be administered via inhalation. These aerosol formulations can be placed into pressurized acceptable propellants, such as dichlorodifluoromethane, propane, and nitrogen. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycols such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl β-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present disclosure. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutical carriers for injectable compositions are well known to those of ordinary skill in the art. See Pharmaceutics and Pharmacy Practice, J.B. Lippincott Co., Philadelphia, Pa., Banker and Chalmers, Eds., 238-250 (1982) and ASHP Handbook on Injectable Drugs, Toissel, 4th ed., 622-630 (1986).

Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, and gels containing, in addition to the active ingredient, such carriers as are known in the art.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Suitable pharmaceutical carriers are described in Remington's Pharmaceutical Sciences, Mack Publishing Company, a standard reference text in this field.

The dose administered to an animal, particularly a human, in the context of the present disclosure should be sufficient to affect a therapeutic response in the animal over a reasonable time frame. One skilled in the art will recognize that dosage will depend upon a variety of factors including a condition of the animal, the body weight of the animal, as well as the severity and stage of the condition being treated.

A suitable dose is that which will result in a concentration of the active agent in a patient which is known to affect the desired response. The preferred dosage is the amount which results in maximum inhibition of the condition being treated, without unmanageable side effects.

The size of the dose also will be determined by the route, timing and frequency of administration as well as the existence, nature, and extend of any adverse side effects that might accompany the administration of the compound and the desired physiological effect.

Useful pharmaceutical dosage forms for administration of the compounds according to the present disclosure can be illustrated as follows:

### Hard Shell Capsules

A large number of unit capsules are prepared by filling standard two-piece hard gelatine capsules each with 100 mg of powdered active ingredient, 150 mg of lactose, 50 mg of cellulose and 6 mg of magnesium stearate.

### Soft Gelatin Capsules

A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

### Tablets

A large number of tablets are prepared by conventional procedures so that the dosage unit was 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

### Immediate Release Tablets/Capsules

These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Moreover, the compounds of the present disclosure can be administered in the form of nose drops, or metered dose and a nasal or buccal inhaler. The drug is delivered from a nasal solution as a fine mist or from a powder as an aerosol.

All publications, patents and patent applications cited in this specification are herein incorporated by reference, and for any and all purpose, as if each individual publication, patent or patent application were specifically and individually indicated to be incorporated by reference. In the case of inconsistencies, the present disclosure will prevail.

The foregoing description of the disclosure illustrates and describes the present disclosure. Additionally, the disclosure shows and describes only the preferred embodiments but, as mentioned above, it is to be understood that the disclosure is capable of use in various other combinations, modifications, and environments and is capable of changes or modifications within the scope of the concept as expressed herein, commensurate with the above teachings and/or the skill or knowledge of the relevant art.

The embodiments described hereinabove are further intended to explain best modes known of practicing it and to enable others skilled in the art to utilize the disclosure in such, or other, embodiments and with the various modifications required by the particular applications or uses. Accordingly, the description is not intended to limit it to the form disclosed herein. Also, it is intended that the appended claims be construed to include alternative embodiments.

What is claimed is:

1. A method for inhibiting DNA methyltransferase in cells of a patient by administering to the patient at least one compound represented by the formulae: 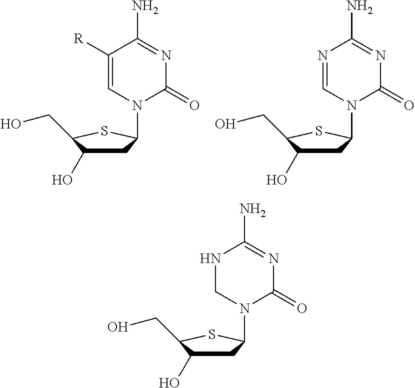
wherein R is individually selected from the group consisting of H, aliphatic acyl, aromatic acyl, halo, alkoxy, alkyl, haloalkyl, alkenyl, haloalkenyl, alkynyl, amino, monoalkylamino, dialkylamino, cyano, aryl and nitro; a pharmaceutically acceptable salt thereof, a prodrug thereof, a solvate thereof or mixtures thereof.

2. The method of claim 1 wherein said at least one compound comprises a compound represented by the formula:

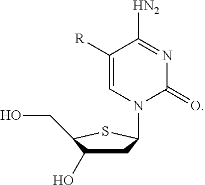

3. The method of claim 2 wherein R is hydrogen.

4. The method of claim 1 wherein said at least one compound comprises a compound represented by the formula:

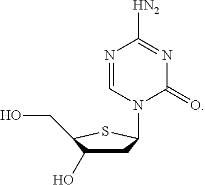

5. The method of claim 1 wherein said at least one compound comprises a compound represented by the formula:

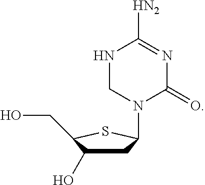

6. The method of claim 1 wherein the patient is afflicted with myeloid dysplastic syndromes or other myeloid malignancies.

7. The method of claim 6 wherein said at least one compound comprises a compound represented by the formula:

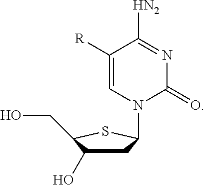

8. The method of claim 7 wherein R is hydrogen.

9. The method of claim 6 wherein said at least one compound comprises a compound represented by the formula:

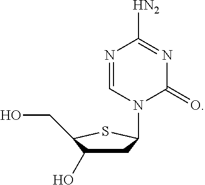

10. The method of claim 6 wherein said at least one compound comprises a compound represented by the formula:

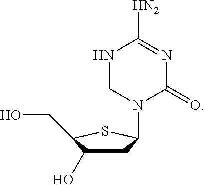

11. A method for treating a patient afflicted with cancers or diseases resulting from or related to aberrant DNA methylation which comprises administering to said patient an effective amount of at least one compound represented by the formula: 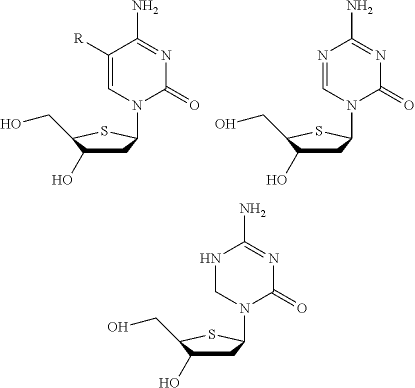
wherein R is individually selected from the group consisting of H, aliphatic acyl, aromatic acyl, halo, alkoxy, alkyl, haloalkyl, alkenyl, haloalkenyl, alkynyl, amino, monoalkylamino, dialkylamino, cyano, aryl and nitro; a pharmaceutically acceptable salt thereof, a prodrug thereof, a solvate thereof and mixtures thereof.

12. The method of claim 11 wherein said at least one compound comprises a compound represented by the formula:

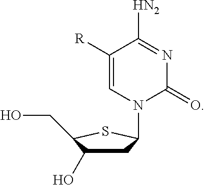

13. The method of claim 12 wherein R is hydrogen.

14. The method of claim 11 wherein said at least one compound comprises a compound represented by the formula:

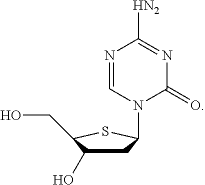

15. The method of claim 11 wherein said at least one compound comprises a compound represented by the formula: [chemical structure]
